(19) United States
(12) Patent Application Publication
(10) Pub. No.: US 2006/0062839 A1
(43) Pub. Date: Mar. 23, 2006

(54) Efficient liposomal encapsulation under mild conditions

(75) Inventors: Alla Polozova, Cranbury, NJ (US); Xingong Li, Cranbury, NJ (US); Walter R Perkins, Pennington, NJ (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Elan Pharmaceuticals, Inc., South San Francisco (CA)

(21) Appl. No.: 10/500,977

(22) PCT Filed: Jan. 8, 2003
(86) PCT No.: PCT/US03/00373

§ 371 Date: Sep. 12, 2005

Related U.S. Application Data
(60) Provisional application No. 60/346,285, filed on Jan. 9, 2002.
(60) Provisional application No. 60/346,285, filed on Jan. 9, 2002.

(51) Int. Cl. A61K 9/127

(52) U.S. Cl. 424/450

(57) ABSTRACT

A method for preparing liposomes containing at least one biologically active substance encapsulated therein under mild conditions comprises the following steps: (A) providing liposomes, wherein the liposomes are prepared by a method other than the instant method; (B) mixing the product of step (A) with a water-miscible organic solvent to form a gel or a liquid containing gel particles; and thereafter (C) (a) mixing the gel or liquid containing gel particles with aqueous medium V to directly form the liposomes containing the at least one biologically active substance encapsulated therein, (b) (i) mixing the gel or liquid containing gel particles with aqueous medium V to form a curd or curdy substance; and (ii) mixing the curd or curdy substance with aqueous medium W to directly form the liposomes containing the at least one biologically active substance encapsulated therein, or (c) (i) cooling the gel or liquid containing gel particles to form a waxy substance; and (ii) mixing the waxy substance with aqueous medium W to directly form the liposomes containing the at least one biologically active substance encapsulated therein; wherein the at least one biologically active substance is added in step (A), (B) or (C), and wherein aqueous media V and W are the same or different.

### FIELD OF THE INVENTION

This invention concerns a method of preparing liposomes containing at least one biologically active substance encapsulated therein, wherein the at least one biologically active substance is encapsulated under mild conditions, and methods of using the liposomes containing the at least one biologically active substance. The method of preparing the liposomes of the present invention has the advantages of being simple, able to generate primarily small liposomes of relatively homogeneous particle size with a high entrapment efficiency and able to encapsulate the biologically active substance without subjecting the biologically active substance to any harsh condition such as high temperatures.

### BACKGROUND OF THE INVENTION

Liposomes are lipid vesicles having at least one aqueous phase completely enclosed by at least one lipid bilayer membrane. Liposomes can be unilamellar or multilamellar. Unilamellar liposomes are liposomes having a single lipid bilayer membrane. Multilamellar liposomes have more than one lipid bilayer membrane with each lipid bilayer membrane separated from the adjacent lipid bilayer membrane by an aqueous layer. The cross sectional view of multilamellar vesicles is often characterized by an onion-like structure.

Liposomes are known to be useful in drug delivery, so many studies have been conducted on the methods of liposome preparation. Descriptions of these methods can be found in numerous reviews (e.g., Szoka et al., “Liposomes: Preparation and Characterization”, in Liposomes: From Physical Structure to Therapeutic Applications, edited by Knight, pp. 51-82, 1981; Deamer et al., “Liposome Preparation: Methods and Mechanisms”, in Liposomes, edited by Ostro, pp. 27-51, 1987; Perkins, “Applications of Liposomes with High Captured Volume”, in Liposomes Rational Design, edited by Janoff, pp. 219-259, 1999).

A method of preparing multilamellar liposome was reported by Bangham et al. (J. Mol. Biol. 13:238-252, 1965). In the method of Bangham et al., phospholipids were mixed with an organic solvent to form a solution. The solution was then evaporated to dryness leaving behind a film of phospholipids on the internal surface of a container. An aqueous medium is added to the container to form multilamellar vesicles (hereinafter referred to as MLVs).

Small unilamellar vesicles (hereinafter referred to as SUVs) were prepared using sonication (Huang, Biochemistry 8:346-352, 1969). A phospholipid was dissolved in an organic solvent to form a solution, which was dried under nitrogen to remove the solvent. An aqueous phase was added to produce a suspension of vesicles. The suspension was sonicated until a clear liquid was obtained, which contained a dispersion of SUVs.

Other methods for the preparation of liposomes were discovered in the 1970s. These methods include the solvent-infusion method, the reverse-phase evaporation method and the detergent removal method. In the solvent-infusion method, a solution of a phospholipid in an organic solvent, most commonly ethanol, was rapidly injected into a larger volume of an aqueous phase under a condition that caused the organic solvent to evaporate. When the organic solvent evaporated upon entry into the aqueous phase, bubbles of the organic solvent's vapor were formed and the phospholipid was left as a thin film at the interface of the aqueous phase and the vapor bubble. As the vapor bubble ascended through the aqueous phase, the phospholipid spontaneously rearranged to form unilamellar and oligolamellar liposomes (e.g., see Batzri et al., Biochim. Biophys. Acta, 298:1015-1019, 1973). Liposomes produced by the solvent-infusion method were mostly unilamellar.

Large unilamellar vesicles (hereinafter referred to as LUVs) were prepared by the reverse-phase evaporation method. In the reverse-phase evaporation method, lipids were dissolved in an organic solvent, such as diethylether, to form a lipid solution. An aqueous phase was added directly into the lipid solution in a ratio of the aqueous phase to the organic solvent of 1:3 to 1:6. The mixture of the lipid/organic solvent/aqueous phase was briefly sonicated to form a homogenous emulsion of inverted micelies. The organic solvent was then removed from the mixture in a two-step procedure, in which the mixture was evaporated at 200-400 mm Hg until the emulsion became a gel, which was then evaporated at 700 mm Hg to remove all the solvent allowing the micelles to coalesce to form a homogeneous dispersion of mainly unilamellar vesicles known as reverse-phase evaporation vesicles (hereinafter referred to as REVs) (e.g., see Papahaduopoulos, U.S. Pat. No. 4,235,871).

In the detergent removal method, a phospholipid was dispersed with a detergent, such as cholate, deoxycholate or Triton X-100, in an aqueous phase to produce a turbid suspension. The suspension was sonicated to become clear as a result of the formation of mixed micelles. The detergent was removed by dialysis or gel filtration to obtain the liposomes in the form of mostly large unilamellar vesicles (e.g., see Enoch et al., Proc. Natl. Acad. Sci. USA, 76:145-149, 1979). The liposomes prepared by the detergent removal method suffer a major disadvantage in the inability to completely remove the detergent, with the residual detergent changing the properties of the lipid bilayer and affecting retention of the aqueous phase.

There were also methods for the preparation of large liposomes involving fusion or budding. These methods generally started with liposomes prepared with another method and disrupted the vesicular structures using mechanical or electrical forces. The disruption induced physical strain in the bilayer structure and changed the hydration and/or surface electrostatics. One of the ways of disrupting the existing vesicular structures was by a freezing and thawing process, which produced vesicle rupture and fusion. The freezing and thawing process increased the size and entrapment volume of the liposome.

Fountain et al. (U.S. Pat. No. 4,588,578) described a method for preparing monophasic lipid vesicles (hereinafter referred to as MPVs), which are lipid vesicles having a plurality of lipid bilayers. MPVs are different from MLVs, SUVs, LUVs and REVs. In the method of Fountain et al., a lipid or lipid mixture and an aqueous phase were added to a water-miscible organic solvent in amounts sufficient to form a monophase. The solvent was then evaporated to form a film. An appropriate amount of the aqueous phase was added to suspend the film, and the suspension was agitated to form the MPVs.

Minchey et al. (U.S. Pat. No. 5,415,867) described a modification of the method of Fountain et al. In the method of Minchey et al., a phospholipid, a water-miscible organic solvent, an aqueous phase and a biologically active agent were mixed to form a cloudy mixture. The solvents in the mixture were evaporated, but not to substantial dryness, under a stream of air in a warm water bath at 37° C. until the mixture formed a monophase, i.e., a clear liquid. As solvent removal continued, the mixture became opaque and gelatinous, in which the gel state indicated that the mixture was hydrated. The purging was continued for 5 minutes to further remove the organic solvent. The gelatinous material was briefly heated at 51° C. until the material liquified. The resulting liquid was centrifuged to form lipid vesicles containing the biologically active agent. The aqueous supernatant was removed and the pellet of lipid vesicles was washed several times. The modification of Minchey et al. was that the biologically active agent and the lipid were maintained as hydrated at all times to avoid the formation of a film of the biologically active agent and lipid upon the complete removal of all the aqueous phase. During evaporation of the organic solvent, the presence of a gel indicated that the monophase was hydrated.

Different techniques were developed to improve the encapsulation efficiency for biologically active compounds. However, little progress has been made to conveniently and efficiently encapsulate molecules, especially large molecules, into small or medium sized liposomes or to devise liposome production to make liposomes of a relatively homogeneous size distribution without resorting to size reduction methodologies (e.g. extrusion and homogenization). The prior art methods of preparing liposomes suffer from some or all of the following problems: being time consuming and not economical, having a low entrapment efficiency and/or generating vesicles of heterogenous size distribution requiring sonication or extrusion to remove large vesicles. The present invention has solved the problems by presenting a new relatively simple method of making liposomes having a high entrapment efficiency and of relatively homogeneous size. In conventional methods of preparing liposomes containing a biologically active substance encapsulated therein, the biologically active substance might be exposed to high temperatures during the preparation of the liposomes and the high temperatures might damage the biologically active substance. In contrast, the new method of the present invention successfully encapsulates a biologically active substance under mild conditions, e.g., without exposing the biologically active substance to high temperatures or solvents that could damage the biologically active substance. This new encapsulation method of the present invention also has advantages of (1) requiring a relatively short preparation time and (2) being operable in a wide range of temperatures.

### SUMMARY OF THE INVENTION

The invention concerns a method for preparing liposomes containing at least one biologically active substance encapsulated therein under mild conditions, said method comprising the following steps:

(A) providing liposomes, wherein the liposomes are prepared by a method other than the instant method;

(B) mixing the product of step (A) with aqueous medium U and a water-miscible organic solvent to form a gel or a liquid containing gel particles; and thereafter

(C) (a) mixing the gel or liquid containing gel particles with aqueous medium V to directly form the liposomes containing the at least one biologically active substance encapsulated therein,
(b) (i) mixing the gel or liquid containing gel particles with aqueous medium V to form a curd or curdy substance; and
   (ii) mixing the curd or curdy substance with aqueous medium W to directly form the liposomes containing the at least one biologically active substance encapsulated therein, or
(c) (i) cooling the gel or liquid containing gel particles to form a waxy substance; and
   (ii) mixing the waxy substance with aqueous medium W to directly form the liposomes containing the at least one biologically active substance encapsulated therein;

wherein the at least one biologically active substance is added in step (A), step (B) and/or step (C), and wherein aqueous media U, V and W are the same or different.

Certain embodiments of the method for the preparation of the liposomes containing the at least one biologically active substance encapsulated under mild conditions of the present invention comprise the following steps:

(A) (a) (i) providing liposomes, wherein the liposomes are prepared by a method other than the instant method; and
(ii) mixing the liposomes of step (A)(a)(i) with the at least one biologically active substance;
(b) (i) providing liposomes in aqueous medium U, wherein the liposomes are prepared by a method other than the instant method; and
   (ii) mixing the liposomes of step (A)(b)(i) with the at least one biologically active substance;
(c) (i) providing liposomes, wherein the liposomes are prepared by a method other than the instant method; and
   (ii) mixing the liposomes of step (A)(c)(i) with aqueous medium U and the at least one biologically active substance;
(d) (i) providing liposomes in aqueous medium U, wherein the liposomes are prepared by a method other than the instant method; and
   (ii) mixing the liposomes of step (A)(d)(i) with aqueous medium U and the at least one biologically active substance;
(e) forming liposomes in the presence of the at least one biologically active substance by a method other than the instant method;
(f) providing liposomes containing the at least one biologically active substance, wherein the liposomes are prepared by a method other than the instant method; or
(g) providing liposomes, wherein the liposomes are prepared by a method other than the instant method;

(B) (a) mixing the product of step (A)(b), (A)(c) or (A)(d) with a water-miscible organic solvent to form a gel or a liquid confining gel particles
(b) mixing the product of step (A)(a), (A)(e) or (A)(f) with aqueous medium U and a water-miscible organic solvent to form a gel or a liquid containing gel particles;
(c) mixing the product of step (A)(g) with aqueous medium U, a water-miscible organic solvent and the at least one biologically active substance to form a gel or a liquid containing gel particles; or
(d) mixing the product of step (A)(g) with aqueous medium U and a water-miscible organic solvent to form a gel or a liquid containing gel particles; and thereafter

(C) (a) mixing the gel or liquid containing gel particles of step (B)(a), (B)(b) or (B)(c) with aqueous medium V to directly form the liposomes containing the at least one biologically active substance encapsulated therein,
(b) (i) mixing the gel or liquid containing gel particles of step (B)(a), (B)(b) or (B)(c) with aqueous medium V to form a curd or curdy substance; and
   (ii) mixing the curd or curdy substance with aqueous medium W to directly form the liposomes containing the at least one biologically active substance encapsulated therein;
(c) (i) cooling the gel or liquid containing gel particles of step (B)(a), (B)(b) or (B)(c) to form a waxy substance;
   (ii) mixing the waxy substance with aqueous medium W to directly form the liposomes containing the at least one biologically active substance encapsulated therein;
(d) mixing the gel or liquid containing gel particles of step (B)(d) with aqueous medium V and the at least one biologically active substance to directly form the liposomes containing the at least one biologically active substance encapsulated therein,
(e) (i) mixing the gel or liquid containing gel particles of step (B)(d) with aqueous medium V and the at least one biologically active substance to form a curd or curdy substance; and
   (ii) mixing the curd or curdy substance with aqueous medium W to directly form the liposomes containing the at least one biologically active substance encapsulated therein;
(f) (i) mixing the gel or liquid containing gel particles of step (B)(d) with aqueous medium V to form a curd or curdy substance; and
   (ii) mixing the curd or curdy substance with aqueous medium W and the at least one biologically active substance to directly form the liposomes containing the at least one biologically active substance encapsulated therein; or
(g) (i) cooling the gel or liquid containing gel particles of step (B)(d) to form a waxy substance;
   (ii) mixing the waxy substance with aqueous medium W and the at least one biologically active substance to directly form the liposomes containing the at least one biologically active substance encapsulated therein;

wherein aqueous media U, V and W are the same or different.

The liposomes provided in step (A)(a)(i), (A)(b)(i), (A)(c)(i), (A)(d)(i), (A)(f) or (A)(g) can be liposomes prepared by any conventional liposome preparation method. Similarly, any conventional liposome preparation method can be used to form the liposomes in step (A)(e).

### DETAILED DESCRIPTION OF THE INVENTION

In certain embodiments of the method of preparing liposomes containing the at least one biologically active substance encapsulated therein under mild conditions of the present invention, a lipid content of the gel or the liquid containing gel particles formed in step (B) is not 15% to 30% by weight of the gel or the liquid containing gel particles.

In certain embodiments of the method of preparing liposomes containing the at least one biologically active substance encapsulated therein under mild conditions of the present invention, a lipid content of the gel or the liquid containing gel particles formed in step (B) is not 15% to 30% by weight of the gel or the liquid containing gel particles and the content of the water-miscible organic solvent in the gel or the liquid containing gel particles is not 14% to 20% by weight of the gel or the liquid containing gel particles.

In certain embodiments of the method of preparing liposomes containing the at least one biologically active substance encapsulated therein under mild conditions of the present invention, the formation of the gel or the liquid containing gel particles in step (B) does not involve the use of any hydrating agent, which is defined as a compound having at least two ionizable groups, one of which ionizable groups is capable of forming an easily dissociative ionic salt, which salt can complex with the ionic functionality of the liposome-forming lipid. The hydrating agent inherently does not form liposomes in and of itself and the hydrating agent must also be physiologically acceptable. Example of the hydrating agent are arginine, homoarginine, γ-aminobutyric acid, glutamic acid, aspartic acid and similar amino acids.

In certain embodiments of the method of preparing liposomes containing the at least one biologically active substance encapsulated therein under mild conditions of the present invention, the gel or the liquid containing gel particles is formed in step (B) without creation of any gas/aqueous phase boundary by sonication or any other method (such as the application of high frequency energy to the mixture of the at least one liposome-forming lipid, the water-miscible organic solvent and aqueous medium Y) of producing a gas/aqueous phase boundary. The “high frequency energy” is the energy having a frequency at least equal to the frequency of ultrasound.

In certain embodiments of the method of preparing liposomes containing the at least one biologically active substance encapsulated therein under mild conditions of the present invention, the liposomes so prepared comprise at least one charged lipid. The at least one charged lipid can be included in the liposomes prepared by any conventional method of liposome preparation provided in step (A)(a)(i), (A)(b)(i), (A)(c)(i) or (A)(d)(i). Alternatively, the at least one charged lipid may be included in the formation of the liposomes in step (A)(e) by any conventional method of liposome preparation. If the at least one charged lipid is added to form the gel or the liquid containing gel particles, the content of the at least one charged lipid in the gel or the liquid containing gel particles can range from about 40% to about 100%, about 50% to about 100%, about 60% to about 100%, about 70% to about 100% or about 80% to about 100% by weight of the lipid(s) in the gel or the liquid containing gel particles. One of the benefits of adding at least one charged lipid in forming the liposomes is that the liposomes formed would have a small size, i.e., a preferred mean diameter, weighted by number, of about 400 nm or less, about 300 nm or less, about 200 nm or less, or about 100 nm or less, without the requirement of any sonication to form the gel or liquid containing gel particles, or the requirement of any sonication or extrusion of the liposomes.

In certain embodiments of the method of preparing liposomes of the present invention, the gel or liquid containing gel particles contains at least one acidic phospholipid, the content of the at least one acidic phospholipid is about 20% to about 100%, about 30% to about 100%, about 40% to about 100%, about 50% to about 100%, about 60% to about 100%, about 70% to about 100% or about 80% to about 100% by weight of the lipid(s) in the gel or liquid containing gel particles.

The method of preparing liposomes under mild conditions of the present invention involves hydration of liposomes prepared by a method other than the method of the present invention (the liposomes are prepared by a method other than the method of the present invention as provided in step (A) of the instant method of the present invention). The liposomes prepared by a method other than the method of the present invention are typically mixed with a water miscible organic solvent to form the gel or the liquid containing gel particles (see step (B) of the method of preparing the liposomes containing the at least one biologically active substance encapsulated therein under mild conditions of the present invention). Hydration of the gel or the liquid containing gel particles leads to direct formation of liposomes without any additional manipulation, such as evaporation or sonication, normally required in prior art methods. Depending on the liposome-forming lipid used, in the mild condition method of the present invention, upon hydration the gel or the liquid containing gel particles may go through a curd or curdy stage, with the formation of a curd or curdy substance, before forming the product of the mild condition method upon further hydration, but no additional manipulation, such as evaporation or sonication, is required other than hydration. For instance, when certain saturated liposome-forming lipids are used in the gel or the liquid containing gel particles, upon hydration of the gel or the liquid containing gel particles go through an intermediate curd or curdy stage, which upon further hydration would directly form the liposomes containing the at least one biologically active substance encapsulated therein without any further manipulation, e.g., sonication or evaporation, required. Alternatively, the gel or the liquid containing gel particles is cooled to obtain a waxy substance, which upon hydration directly forms the liposomes containing the at least one biologically active substance encapsulated therein under mild conditions, without any further manipulation, such as evaporation or sonication, required.

Within the scope of the present invention, the method of preparing liposomes of the invention can be used to encapsulate at least one biologically active substance in the liposomes under mild conditions. The at least one biologically active substance to be encapsulated can be, if it is hydrophobic, dissolved in the water-miscible organic solvent or, if it is hydrophilic, dissolved in an aqueous medium, preferably at a high concentration. To form the gel or the liquid containing gel particles in step (B) of the mild condition method of the present invention, the liposomes of step (A) and the water-miscible organic solvent is mixed with an appropriate volume of aqueous medium to form the gel or the liquid containing gel particles. In step (B), the amount of lipid in the liposomes mixed with the aqueous medium and the water-miscible organic solvent to form the gel or the liquid containing gel particles can range from 1% by weight of the gel or the liquid containing gel particles to the hydration limit of the lipid in water. The “hydration limit” is the maximum amount of lipid in a given amount of water that would keep the lipid in a liposomal state. The amount of lipid in the liposomes mixed with the aqueous medium and the water-miscible organic solvent to form the gel or the liquid containing gel particles in step (B) can range from about 5% to about 95%, about 10% to about 95%, about 15% to about 95%, about 20% to about 95%, about 30% to about 95%, about 40% to about 95%, about 50% to about 95%, about 60% to about 95%, or about 70% to about 95% by weight of the gel or the liquid containing gel particles. The amount of lipid in the liposomes mixed with the aqueous medium and the water-miscible organic solvent to form the gel or the liquid containing gel particles in step (B) can also range from about 5% to about 90%, about 10% to about 90%, about 15% to about 90%, about 20% to about 90%, about 30% to about 90%, about 40% to about 90%, about 50% to about 90%, about 60% to about 90%, or about 70% to about 90% by weight of the gel or the liquid containing gel particles. In step (B), the amount of lipid in the liposomes mixed with the aqueous medium and the water-miscible organic solvent to form the gel or the liquid containing gel particles can also range from about 5% to about 85%, about 10% to about 85%, about 15% to about 85%, about 20% to about 85%, about 30% to about 85%, about 40% to about 85%, about 50% to about 85%, about 60% to about 85%, or about 70% to about 85% by weight of the gel or the liquid containing gel particles. Alternatively, the amount of lipid in the liposomes mixed with the aqueous medium and the water-miscible organic solvent to form the gel or the liquid containing gel particles in step (B) can range from about 5% to about 80%, about 10% to about 80%, about 15% to about 80%, about 20% to about 80%, about 30% to about 80%, about 40% to about 80%, about 50% to about 80%, about 60% to about 80%, about 70% to about 80%, about 10% to about 70%, about 20% to about 60%, or about 30% to about 50% by weight of the gel or the liquid containing gel particles. The amount of the lipid is preferably from about 45% to about 80%, more preferably about 30% to about 50% by weight of the water-miscible organic solvent. For instance, the amount of the lipid can be about 40% or 45% by weight of the water-miscible organic solvent.

In step (C) of the method of preparing the liposomes containing the at least one biologically active substance encapsulated therein under mild conditions, aqueous medium V is preferably mixed with the gel or the liquid containing gel particles in increments. The size of the increment can be up to about 1000%, up to about 500%, up to about 200%, up to about 100%, up to about 80%, up to about 60%, up to about 50%, up to about 40%, up to about 30%, up to about 20%, or up to about 10% of the weight of the gel or the liquid containing gel particles before the gel or the liquid is mixed with any aqueous medium V. The size of the increment is preferably up to about 5%, up to about 4%, up to about 3%, up to about 2% or up to about 1% of the weight of the gel or the liquid containing gel particles before the gel or the liquid is mixed with any aqueous medium V. The size of the increment can alternatively be up to about 0.5% or up to about 0.1% of the weight of the gel or the liquid containing gel particles before the gel or the liquid is mixed with any aqueous medium V. The size of the increment can also be from about 0.001% to about 10%, from about 0.001% to about 5%, from about 0.001% to about 1% or from about 0.001% to about 0.1% of the weight of the gel or the liquid containing gel particles before the gel or the liquid is mixed with any aqueous medium V.

The aqueous medium U, aqueous medium V and/or aqueous medium W is preferably an aqueous buffer. Examples of the aqueous buffer include citrate buffer, Tris buffer, phosphate buffer and a oufer conta mg sucrose or dextrose.

In step (C) of the method of the present invention, the gel or the liquid containing gel particles and aqueous medium V are mixed by either adding aqueous medium V to the gel, or adding or infusing the gel or the liquid containing gel particles into aqueous medium V.

The expression, “to directly form the liposomes containing the at least one biologically active substance encapsulated therein”, means that no additional procedure or manipulation, such as evaporation or sonication, other than the potential intermediate formation of the waxy substance if the gel or the liquid containing gel particles is cooled (the hydration of the waxy substance would directly lead to the liposomes without any further manipulation or procedure such as evaporation or sonication required) or potential intermediate step of the formation of the curd or curdy substance if certain lipids are used (the hydration of the curd or curdy substance would directly result in the liposomes without any further manipulation or procedure such as evaporation or sonication required), is required for the formation of the liposomes having the at least one biologically active substance encapsulated under mild conditions.

The liposomes of step (A) can be formed by any conventional liposome preparation method. Preferably, the liposomes of step (A) comprise a phosphatidylcholine, e.g., dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine and 2-palmitoyl-1-oleoyl-sn-glycero-3-phosphocholine, or N-acyl phosphatidylethanolamine, e.g., 1,2-dioleoyl-sn-glycero-N-dodecanoyl-3-phosphoethanolamine.

The liposomes of step (A) can further comprise a fusogenic lipid (see Meers et al, U.S. Pat. No. 6,120,797, the disclosure of which is herein incorporated by reference). Examples of fusogenic lipid are N-acyl phosphatidylethanolamine, such as N-decanoyl phosphatidylethanolamine, N-undecanoyl phosphatidylethanoiarnine, N-dodecanoyl phosphatidylethanolamine, N-tridecanoyl phosphatidylethanolamine, and N-tetradecanoyl phosphatidylethanolamine. N-acyl phosphatidylethanolamine that can be used include 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-ipalmitoyl-sn-glycero-N-dodecanoyl-3-phosphoethanolamine, 1,2-dipalmitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3-phosphoethanolamine, 1-oleoyl-2-palmitoyl-sn-glycero-N-tetradecanoyl-3-phosphoethanolamine, 1-palirtoyl-2-oleoyl-sn-glycero-N-decanoyl-3-phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine. The fusogenicity-increasing N-acyl phosphatidylethanolamine is preferably N-dodecanoyl phosphatidylethanolamine and more preferably 1,2-dioleoyl-sn-glycero-N-dodecanoyl-3-phosphoethanolamine.

The liposomes step (A) of the method of the present invention can further comprise a sterol. Preferably, the sterol is cholesterol.

Certain embodiments of the preparatory methods of the present invention use one, or a combination (at any ratio), of the following lipids: phosphatidylcholines, phosphatidylglycerols, phosphatidylserines, phosphatidylethanolamines, phosphatidylinositols, headgroup modified phospholipids, headgroup modified phosphatidylethanolamines, lyso-phospholipids, phosphocholines (ether linked lipids), phosphoglycerols (ether linked lipids), phosphoserines (ether linked lipids), phosphoethanolamines (ether linked lipids), sphingomyelins, sterols, such as cholesterol hemisuccinate, tocopherol hemisuccinate, ceramides, cationic lipids, monoacyl glycerol, diacyl glycerol, triacyl glycerol, fratty acids, fratty acid methyl esters, single-chain nonionic lipids, glycolipids, lipid-peptide conjugates and lipid-polymer conjugates. However, in certain embodiments of the method of preparing the liposomes of the present invention having the at least one biologically active substance encapsulated therein under mild conditions, no phosphatidylcholine is used. The lipid or a combination thereof are included in the starting liposome, included in the gel or the liquid containing gel particles, added to the gel or the liquid containing gel particles or added during the hydration of the gel or the liquid containing gel particles in the methods of preparing the liposomes having the at least one biologically active substance encapsulated therein under mild conditions of the present invention. In the method of preparing the liposomes having the at least one biologically active substance encapsulated therein of the present invention, these lipids can be added when the starting liposomes of step (A) are prepared with a method other than the instant method, added in step (A), added in step (B), or added in both steps (A) and (B).

In certain embodiments of the method of preparing liposomes encapsulating the at least one biologically active substance under mild conditions of the present invention, at least one charged lipid is added when the starting liposomes of step (A) are prepared with a method other than the instant method, added in step (A), added in step (B), or added in both steps (A) and (B). The “charged lipid” is a lipid having a net negative or positive charge in the molecule. Examples of the charged lipid include N-acyl phosphatidylethanolamine, phosphatidylserine, phosphatidylinositol, phosphatidylglycerol, diphosphatidylglycerol (i.e., cardiolipin) and phosphatidic acid. The at least one “charged lipid” can be liposome forming.

In the method of the present invention, the “water-miscible organic solvent” is an organic solvent that, when mixed with water, forms a homogeneous liquid, i.e., with one phase. The water-miscible organic solvent can be selected from the group consisting of acetaldenyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline. Acetonitrile, C₁-C₃ alcohols and acetone are preferred examples of the water-miscible organic solvent. If a C₁-C₃ alcohol is used as the water-miscible organic solvent, it is preferably methanol, ethanol, 1-propanol, 2-propanol, ethylene glycol and propylene glycol. The C₁-C₃ alcohols are more preferably ethanol, 1-propanol or 2-propanol, with ethanol being the most preferred.

One of the advantages of the method of the present invention is that an organic solvent, such as ethanol, of relatively low toxicity can be used. With a water-miscible organic solvent of relatively low toxicity, the liposomes prepared according to the method of the present invention would not be expected to pose any significant toxicity hazard even when the liposomes contain a residual amount of the water-miscible organic solvent.

In one of the embodiments of the method of preparing liposomes of the present invention, after step (C) the liposomes containing the at least one biologically active substance are washed with an aqueous medium by centrifugation, gel filtration or dialysis.

Liposomes are useful as delivery vehicles of encapsulated substances. The method of the present invention can be used to encapsulate at least one biologically active substance in liposomes. The liposomes containing the at least one biologically active substance encapsulated therein prepared by the method of the present invention have the advantages of a high entrapment efficiency and a relatively homogeneous particle size. Due to the simplicity of the procedures, the method of preparing the liposomes of the present invention allows relatively rapid production of the liposomes at a low cost. The method of the present invention has the additional advantage of being easily controlled and modified, e.g., by selecting a batch or continuous operation, or by choosing the appropriate temperature at which the method is conducted, to fit the special requirements of different formulations.

The at least one biologically active substance encapsulated in the liposomes prepared by the method of the present invention includes a pharmaceutical agent, nucleic acid, protein, peptide, diagnostic agent, antigen and hapten, especially an antigenic substance or hapten structurally sensitive to dehydration (e.g., solvent exposure at an air to water interface). The “antigen” that can be encapsulated includes toxoids. The “antigenic substance or hapten structurally sensitive to dehydration” is an antigenic substance or hapten that loses structural integrity upon an exposure to dehydration, e.g., in an air to water interface. Examples of the “antigenic substance or hapten structurally sensitive to dehydration” are certain toxoids, e.g., tetanus toxoids.

Examples of the pharmaceutical agent that can be encapsulated in the liposomes are anti-neoplastic agents, anti-microbial agents, anti-viral agents, antihypertensive agents, anti-inflammatory agents, bronchodilators, local anesthetics and immunosuppressants. Examples of the pharmaceutical agent include doxorubicin (anti-neoplastic agent), macrolide antibiotics (anti-bacterial agent), amphotericin B (anti-fungal agent) and cyclosporin (immunosuppressant). Since systemic delivery of hydrophobic pharmaceutical agents is usually a problem due to the poor water solubility of the agents, liposomes are especially useful as delivery vehicles for hydrophobic pharmaceutical agents because the liposomes contain a significant amount of lipids with which the hydrophobic pharmaceutical agents can associate. As a result, the at least one pharmaceutical agent to be encapsulated in the liposomes prepared by the method of the present invention is preferably hydrophobic. For instance, bioactive lipids are especially suited for encapsulation in the liposomes prepared by the method of the present invention.

The at least one biologically active substance that is encapsulated in the liposomes prepared by the method of the present invention can be a diagnostic agent. Examples of the diagnostic agents include dyes, radioactive diagnostic agents and antibodies.

The at least one biologically active substance can also be a protein, such as an antibody, proteinaceous antigen, enzyme, cytochrome C, cytokine, toxin (e.g., tetanoid toxin) and transcription factor.

In some of the embodiments of the present invention, the at least one biologically active substance is a nucleic acid, including oligonucleotide, RNA and DNA. The oliogonucleotide that can be encapsulated can be of a size of from about 5 to about 500 bases. Examples of RNA that can be encapsulated in the liposomes prepared according to the present invention are anti-sense RNA and RNA interference or RNAᵢ. The DNA that can be encapsulated in the liposomes prepared according to the present invention includes a plasmid DNA. The plasmid DNA can be of up to 20 kb, up to 15 kb, up to 10 kb, from about 0.5 kb to about 20 kb, from about 1 kb to about 15 kb, from about 2 kb to about 10 kb or from about 3 kb to about 7 kb in size.

Liposomes prepared by the mild condition method of the present invention containing the plasmid DNA are useful in gene therapy, transfection of eukaryotic cells and transformation of prokaryotic cells. An aspect of the invention is a method for transfecting cells, preferably mammalian cells such as human cells, said method comprising contacting the cells in vivo or in vitro with the liposomes prepared containing the plasmid DNA encapsulated under mild conditions as prepared by the method of the present invention, wherein the plasmid DNA preferably contains a gene of interest. The transfection method is also useful in a method for gene therapy comprising contacting target cells of a subject in need of the gene therapy with the liposomes containing the plasmid DNA encapsulated under mild conditions, in vitro (e.g., via incubation) or in vivo (e.g., via administration of the liposomes into the subject), wherein the plasmid DNA contains a gene having the desired therapeutic effect on the subject. Within the scope of the invention is a method of transforming prokaryotic cells comprising contacting (e.g., via incubation) the prokaryotic cells with the liposomes containing a plasmid DNA encapsulated therein under mild conditions prepared by the method of the present invention to obtain transformation of the prokaryotic cells.

The liposomes containing the at least one biologically active substance encapsulated therein prepared by the method of the present invention can further comprise a targeting agent to facilitate the delivery of the at least one biologically active substanct to a proper target in a biological system. Examples of the targeting agent include antibodies, a molecule containing biotin, a molecule containing streptavidin, or a molecule containing a folate or transferrin molecule.

The liposomes prepared by the method of the present invention having at least one biologically active substance encapsulated therein can be administered to a subject in need of the at least one biologically active substance via an oral or parenteral route (e.g., intravenous, intramuscular, intraperitoneal, subcutaneous and intrathecal routes) for therapeutic or diagnostic purposes. The dose of the liposomes to be administered is dependent on the at least one biologically active substance involved, and can be adjusted by a person skilled in the art based on the health of the subject and the medical condition to be treated or diagnosed. For diagnostic purposes, some the liposomes of the present invention can be used in vitro.

Some aspects of the present invention are shown in the following working example. However, the scope of the present invention is not to be limited by the working example. A person skilled in the art can practice the present invention as recited in the claims beyond the breadth of the working example. The working example is provided for illustration purposes only.

Certain abbreviations were used for the names of some of the lipids employed in the working example:
1,2-Distearoyl-sn-Glycero-3-Phosphocholine (DSPC) and
1,2-Distearoyl-sn-Glycero-3-[Phospho-rac-(1-glycerol)] (DSPG).

### EXAMPLE 1

### DSPC-DSPG-Cholesterol, 4:2:4

The mild-condition method of the present invention was demonstrated in this experiment. Amounts of 8 mg DSPC, 4 mg of DSPG, 3.9 mg of cholesterol and 0.03 mg of NBD-PE were dissolved in 2 ml of a chloroform-methanol (1:1) solvent mixture and placed in a glass test tube. The solvent mixture was evaporated under a stream of nitrogen at 50° C. The resultant lipid film was dried under oil pump vacuum for 3 hours and then hydrated with the addition of 80 μl of a 100 mM Tris buffer, pH 7, at 50° C. forming liposomes. An amount of 1.2 μl of 330 mM sulforhodamine 101 (SR101) solution was added to the sample to generate a SR101 concentration of 5 mM in the sample. Then 120 mg of ethanol was rapidly injected into the sample upon vigorous vortexing to transform the liposomal suspension into an emulsion of soft gel particles. Immediately after the addition of ethanol, 2 ml of the 100 mM Tris buffer were quickly mixed with the sample in 100 μl increments upon rigorous vortexing. The sample was transformed back to a liposome suspension. The sample was then dialyzed against 0.5 L of buffer for 12 hours with one buffer change. The resultant liposomes possessed the following properties: the liposomes (a) captured 80% of the added SR101 as determined from SR101 fluorescence, (b) had an average diameter of 110 nm as measured by dynamic light scattering, and (c) had 50% of the total lipid residing on the outer liposomal shell as determined by NBD-PE dithionite reduction lamellarity assay indicating that the liposomes were mostly unilamellar.

What is claimed is:

1. A method for preparing liposomes containing at least one biologically active substance encapsulated therein under mild conditions, said method comprising the following steps:
(A) providing liposomes, wherein the liposomes are prepared by a method other than the instant method;
(B) mixing the product of step (A) with aqueous medium U and a water-miscible organic solvent to form a gel or a liquid containing gel particles; and thereafter
(C) (a) mixing the gel or liquid containing gel particles with aqueous medium V to directly form the liposomes containing the at least one biologically active substance encapsulated therein,
(b) (i) mixing the gel or liquid containing gel particles with aqueous medium V to form a curd or curdy substance; and
(ii) mixing the curd or curdy substance with aqueous medium W to directly form the liposomes containing the at least one biologically active substance encapsulated therein, or
(c) (i) cooling the gel or liquid containing gel particles to form a waxy substance; and
(ii) mixing the waxy substance with aqueous medium W to directly form the liposomes containing the at least one biologically active substance encapsulated therein;
wherein the at least one biologically active substance is added in step (A), step (B) and/or step (C), and wherein aqueous media U, V and W are the same or different.

2. A method for preparing liposomes containing at least one biologically active substance encapsulated therein under mild conditions, said method comprising the following steps:
(A) (a) (i) providing liposomes, wherein the liposomes are prepared by a method other than the instant method; and
(ii) mixing the liposomes of step (A)(a)(i) with the at least one biologically active substance;
(b) (i) providing liposomes in aqueous medium U, wherein the liposomes are prepared by a method other than the instant method; and
(ii) mixing the liposomes of step (A)(b)(i) with the at least one biologically active substance;
(c) (i) providing liposomes, wherein the liposomes are prepared by a method other than the instant method; and
(ii) mixing the liposomes of step (A)(c)(i) with aqueous medium U and the at least one biologically active substance;
(d) (i) providing liposomes in aqueous medium U, wherein the liposomes are prepared by a method other than the instant method; and
(ii) mixing the liposomes of step (A)(d)(i) with aqueous medium U and the at least one biologically active substance;
(e) forming liposomes in the presence of the at least one biologically active substance by a method other than the instant method;
(f) providing liposomes containing the at least one biologically active substance, wherein the liposomes are prepared by a method other than the instant method; or
(g) providing liposomes, wherein the liposomes are prepared by a method other than the instant method;
(B) (a) mixing the product of step (A)(b), (A)(c) or (A)(d) with a water-miscible organic solvent to form a gel or a liquid containing gel particles;
(b) mixing the product of step (A)(a), (A)(e) or (A)(f) with aqueous medium U and a water-miscible organic solvent to form a gel or a liquid containing gel particles;
(c) mixing the product of step (A) (g) with aqueous medium U, a water-miscible organic solvent and the at least one biologically active substance to form a gel or a liquid containing gel particles; or
(d) mixing the product of step (A) (g) with aqueous medium U and a water-miscible organic solvent to form a gel or a liquid containing gel particles; and thereafter
(C) (a) mixing the gel or liquid containing gel particles of step (B)(a), (B)(b) or (B)(c) with aqueous medium V to directly form the liposomes containing the at least one biologically active substance encapsulated therein,
(b) (i) mixing the gel or liquid containing gel particles of step (B)(a), (B)(b) or (B)(c) with aqueous medium V to form a curd or curdy substance; and
(ii) mixing the curd or curdy substance with aqueous active substance encapsulated therein;
(c) (i) cooling the gel or liquid containing gel particles of step (B)(a), (B)(b) or (B)(c) to form a waxy substance;
(ii) mixing the waxy substance with aqueous medium W to directly form the liposomes containing the at least one biologically active substance encapsulated therein;
(d) mixing the gel or liquid containing gel particles of step (B)(d) with aqueous medium V and the at least one biologically active substance to directly form the liposomes containing the at least one biologically active substance encapsulated therein,
(e) (i) mixing the gel or liquid containing gel particles of step (B)(d) with aqueous medium V and the at least one biologically active substance to form a curd or curdy substance; and
(ii) mixing the curd or curdy substance with aqueous medium W to directly form the liposomes containing the at least one biologically active substance encapsulated therein;
(f) (i) mixing the gel or liquid containing gel particles of step (B)(d) with aqueous medium V to form a curd or curdy substance; and
(ii) mixing the curd or curdy substance with aqueous medium W and the at least one biologically active substance to directly form the liposomes containing the at least one biologically active substance encapsulated therein; or
(g) (i) cooling the gel or liquid containing gel particles of step (B)(d) to form a waxy substance;
(ii) mixing the waxy substance with aqueous medium W and the at least one biologically active substance to directly form the liposomes containing the at least one biologically active substance encapsulated therein;
wherein aqueous media U, V and W are the same or different.

3. The method of claim 2, wherein the liposomes containing the at least one biologically active substance encapsulated therein of step (C) are washed with an aqueous medium by centrifugation, gel filtration or dialysis.

4. The method of claim 2, wherein the organic solvent is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

5. The method of claim 4, wherein the organic solvent is acetonitrile, acetone or a C₁-C₃ alcohol.

6. The method of claim 5, wherein the organic solvent is methanol, ethanol, 1-propanol, 2-propanol, ethylene glycol or propylene glycol.

7. The method of claim 6, wherein the organic solvent is ethanol, 1-propanol or 2-propanol.

8. The method of claim 7, wherein the organic solvent is ethanol.

9. The method of claim 4, wherein the organic solvent is acetone.

10. The method of claim 2, wherein aqueous medium U, aqueous medium V and/or aqueous medium W is an aqueous buffer.

11. The method of claim 2, wherein the gel or liquid and aqueous medium V are mixed in step (C) by adding aqueous medium V to the gel or the liquid containing gel particles.

12. The method of claim 2, wherein the gel or the liquid containing gel particles and aqueous medium V are mixed in step (C) by adding or infusing the gel or liquid into aqueous medium V.

13. The method of claim 2, wherein the at least one biologically active substance is a nucleic acid, pharmaceutical agent, diagnostic agent, protein, peptide, antigen, cytochrome C, transcription factor, cytokine or hapten.

14. The method of claim 13, wherein the at least one biologically active substance is a plasmid DNA.

15. The method of claim 14, wherein the plasmid DNA is up to about 20 kb in size.

16. The method of claim 15, wherein the plasmid DNA is of from about 0.5 kb to about 20 kb in size.

17. The method of claim 16, wherein the plasmid DNA is of about 1 kb to about 15 kb in size.

18. The method of claim 17, wherein the plasmid DNA is of about 2 kb to about 10 kb in size.

19. The method of claim 18, wherein the plasmid DNA is of about 3 kb to about 7 kb in size.

20. The method of claim 2, wherein the at least one biologically active substance is selected from the group consisting of proteins and antigens structurally sensitive to dehydration.

21. The method of claim 20, wherein the proteins and antigens structurally sensitive to dehydration are tetanus toxoids.

22. The method of claim 2, wherein the at least one biologically active substance is at least one pharmaceutical agent selected from the group consisting of anti-neoplastic agents, anti-microbial agents, anti-viral agents, antihypertensive agents, anti-inflammatory agents, bronchodilators, local anesthetics and immunosuppressants.

23. The method of claim 22, wherein the at least one pharmaceutical agent is selected from the group consisting of anti-bacterial agents and anti-fungal agents.

24. The method of claim 22, wherein the at least one pharmaceutical agent is selected from the group consisting of anti-fungal agents and anti-neoplastic agents.

25. The method of claim 2, wherein the at least one biologically active substance is a bioreactive lipid.

26. The method of claim 2, wherein the at least one biologically active substance is an antibody, enzyme or cytokine.

27. The method of claim 2, wherein the at least one biologically active substance is an RNA.

28. The method of claim 2, wherein the at least one biologically active substance is an oligonucleotide.

29. The method of claim 28, wherein the at least one biologically active substance is an oligonucleotide of about 5 to about 500 bases in size.

30. The method of claim 2, wherein the liposome of step (A) further comprises a sterol.

31. The method of claim 30, wherein the sterol is cholesterol.

32. A method for preparing liposomes containing at least one biologically active substance encapsulated therein under mild conditions, said method comprising the following steps:
(A) (a) (i) providing liposomes, wherein the liposomes are prepared by a method other than the instant method; and
(ii) mixing the liposomes of step (A)(a)(i) with the at least one biologically active substance;
(b) (i) providing liposomes in aqueous medium U, wherein the liposomes are prepared by a method other than the instant method; and
(ii) mixing the liposomes of step (A)(b)(i) with the at least one biologically active substance;
(c) (i) providing liposomes, wherein the liposomes are prepared by a method other than the instant method; and
(ii) mixing the liposomes of step (A)(c)(i) with aqueous medium U and the at least one biologically active substance;
(d) (i) providing liposomes in aqueous medium U, wherein the liposomes are prepared by a method other than the instant method; and
(ii) mixing the liposomes of step (A)(d)(i) with aqueous medium U and the at least one biologically active substance;
(e) forming liposomes in the presence of the at least one biologically active substance by a method other than the instant method;
(f) providing liposomes containing the at least one biologically active substance, wherein the liposomes are prepared by a method other than the instant method; or
(g) providing liposomes, wherein the liposomes are prepared by a method other than the instant method;
(B) (a) mixing the product of step (A)(b), (A)(c) or (A)(d) with a water-miscible organic solvent to form a gel or a liquid containing gel particles;
(b) mixing the product of step (A)(a), (A)(e) or (A)(f) with aqueous medium U and a water-miscible organic solvent to form a gel or a liquid containing gel particles;
(c) mixing the product of step (A)(g) with aqueous medium U, a water-miscible organic solvent and the at least one biologically active substance to form a gel or a liquid containing gel particles; or
(d) mixing the product of step (A)(g) with aqueous medium U and a water-miscible organic solvent to form a gel or a liquid containing gel particles; and thereafter
(C) (a) mixing the gel or liquid containing gel particles of step (B)(a), (B)(b) or (B)(c) with aqueous medium V to directly form the liposomes containing the at least one biologically active substance encapsulated therein,
(b) (i) mixing the gel or liquid containing gel particles of step (B)(a), (B)(b) or (B)(c) with aqueous medium V to form a curd or curdy substance; and
(ii) mixing the curd or curdy substance with aqueous medium W to directly form the liposomes containing the at least one biologically active substance encapsulated therein;
(c) (i) cooling the gel or liquid containing gel particles of step (B)(a), (B)(b) or (B)(c) to form a waxy substance;
(ii) mixing the waxy substance with aqueous medium W to directly form the liposomes containing the at least one biologically active substance encapsulated therein;
(d) mixing the gel or liquid containing gel particles of step (B)(d) with aqueous medium V and the at least one biologically active substance to directly form the liposomes containing the at least one biologically active substance encapsulated therein,
(e) (i) mixing the gel or liquid containing gel particles of step (B)(d) with aqueous medium V and the at least one biologically active substance to form a curd or curdy substance; and
(ii) mixing the curd or curdy substance with aqueous medium W to directly form the liposomes containing the at least one biologically active substance encapsulated therein;
(f) (i) mixing the gel or liquid containing gel particles of step (B)(d) with aqueous medium V to form a curd or curdy substance; and
(ii) mixing the curd or curdy substance with aqueous medium W and the at least one biologically active substance to directly form the liposomes containing the at least one biologically active substance encapsulated therein; or
(g) (i) cooling the gel or liquid containing gel particles of step (B)(d) to form a waxy substance;
(ii) mixing the waxy substance with aqueous medium W and the at least one biologically active substance to directly form the liposomes containing the at least one biologically active substance encapsulated therein;
wherein aqueous media U, V and W are the same or different and a phospholipid content of the gel or the liquid containing gel particles is not 15 to 30% by weight of the gel or the liquid containing gel particles.

33. The method of claim 32, wherein step (C)(a) or step (C)(b)(i) is conducted by mixing the gel or liquid with aqueous medium V and the at least one biologically active substance, and/or step (C)(b)(ii) is conducted by mixing the curd or curdy substance with aqueous medium W and the at least one biologically active substance.

34. The method of claim 32, wherein the liposomes containing the at least one biologically active substance encapsulated therein of step (C) are washed with an aqueous medium by centrifugation, gel filtration or dialysis.

35. The method of claim 32, wherein the organic solvent is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methyhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

36. The method of claim 35, wherein the organic solvent is methanol, ethanol, 1-propanol, 2-propanol, ethylene glycol or propylene glycol.

37. The method of claim 36, wherein the organic solvent is ethanol, 1-propanol or 2-propanol.

38. The method of claim 37, wherein the organic solvent is ethanol.

39. The method of claim 35, wherein the organic solvent is acetonitrile or acetone.

40. The method of claim 32, wherein aqueous medium U, aqueous medium V and/or aqueous medium W is an aqueous buffer.

41. The method of claim 32, wherein the gel or liquid and aqueous medium V are mixed in step (C) by adding aqueous medium V to the gel or the liquid containing gel particles.

42. The method of claim 32, wherein the gel or the liquid containing gel particles and aqueous medium V are mixed in step (C) by adding or infusing the gel or liquid into aqueous medium V.

43. The method of claim 32, wherein the at least one biologically active substance is a nucleic acid, protein, peptide, enzyme, cytochrome C, transcription factor, cytokine, antigen, hapten, pharmaceutical agent or diagnostic agent.

44. The method of claim 43, wherein the at least one biologically active substance is a plasmid DNA.

45. The method of claim 44, wherein the plasmid DNA is up to about 20 kb in size.

46. The method of claim 45, wherein the plasmid DNA is of from about 0.5 kb to about 20 kb in size.

47. The method of claim 46, wherein the plasmid DNA is of about 1 kb to about 15 kb in size.

48. The method of claim 47, wherein the plasmid DNA is of about 2 kb to about 10 kb in size.

49. The method of claim 48, wherein the plasmid DNA is of about 3 kb to about 7 kb in size.

50. The method of claim 32, wherein the at least one biologically active substance is selected from the group consisting of proteins and antigens structurally sensitive to dehydration.

51. The method of claim 50, wherein the at least one biologically active substance is a tetanus toxoid.

52. The method of claim 32, wherein the at least one biologically active substance is at least one pharmaceutical agent selected from the group consisting of anti-neoplastic agents, anti-microbial agents, anti-viral agents, antihypertensive agents, anti-inflammatory agents, bronchodilators, local anesthetics and immunosuppressants.

53. A method for preparing liposomes containing at least one biologically active substance encapsulated therein under mild conditions, said method comprising the following steps:
(A) (a) (i) providing liposomes, wherein the liposomes are prepared by a method other than the instant method; and
(ii) mixing the liposomes of step (A)(a)(i) with the at least one biologically active substance;
(b) (i) providing liposomes in aqueous medium U, wherein the liposomes are prepared by a method other than the instant method; and
(ii) mixing the liposomes of step (A)(b)(i) with the at least one biologically active substance;
(c) (i) providing liposomes, wherein the liposomes are prepared by a method other than the instant method; and
(ii) mixing the liposomes of step (A)(c)(i) with aqueous medium U and the at least one biologically active substance;
(d) (i) providing liposomes in aqueous medium U, wherein the liposomes are prepared by a method other than the instant method; and
(ii) mixing the liposomes of step (A)(d)(i) with aqueous medium U and the at least one biologically active substance;
(e) forming liposomes in the presence of the at least one biologically active substance by a method other than the instant method; or
(f) forming liposomes in aqueous medium U in the presence of the at least one biologically active substance by a method other than the instant method;
(B) (a) mixing the product of step (A)(b), (A)(c), (A)(d) or (A)(f) with a water-miscible organic solvent to form a gel or a liquid containing gel particles; or
(b) mixing the product of step (A)(a) or (A)(e) with aqueous medium U and a water-miscible organic solvent to form a gel or a liquid containing gel particles; and thereafter
(C) (a) mixing the gel or liquid containing gel particles with aqueous medium V to directly form the liposomes containing the at least one biologically active substance encapsulated therein, or
(b) (i) mixing the gel or liquid containing gel particles with aqueous medium V to form a curd or curdy substance; and
(ii) mixing the curd or curdy substance with aqueous medium W to directly form the liposomes containing the at least one biologically active substance encapsulated therein,
wherein aqueous media U, V and W are the same or different and the gel or the liquid containing gel particles contain no hydrating agent.

54. The method of claim 53, wherein step (C)(a) or step (C)(b)(i) is conducted by mixing the gel or liquid with aqueous medium V and the at least one biologically active substance, and/or step (C)(b)(ii) is conducted by mixing the curd or curdy substance with aqueous medium W and the at least one biologically active substance.

55. The method of claim 53, wherein the liposomes containing the at least one biologically active substance encapsulated therein of step (C) are washed with an aqueous medium by centrifugation, gel filtration or dialysis.

56. The method of claim 53, wherein the organic solvent is selected from the group consisting of acetaldehyde, acetone, acetontrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl)ether, bis(2-hydroxyethyl)ether, bis(2-hydropropyl)ether, bis(2-methoxyethyl)ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

57. The method of claim 56, wherein the organic solvent is acetonitrile, acetone or a C₁-C₃ alcohol.

58. The method of claim 57, wherein the organic solvent is methanol, ethanol, 1-propanol, 2-propanol, ethylene glycol or propylene glycol.

59. The method of claim 58, wherein the organic solvent is ethanol, 1-propanol or 2-propanol.

60. The method of claim 59, wherein the organic solvent is ethanol.

61. The method of claim 56, wherein the organic solvent is acetone.

62. The method of claim 53, wherein aqueous medium U, aqueous medium V and/or aqueous medium W is an aqueous buffer.

63. The method of claim 2, wherein the liposomes of step (A) comprise at least one fusogenic lipid.

64. The method of claim 63, wherein the at least one fusogenic lipid is selected from the group consisting of N-acyl phosphatidylethanolamine.

65. The method of claim 64, wherein the at least one fusogenic lipid is selected from the group consisting of N-decanoyl phosphatidylethanolamine, N-dodecanoyl phosphatidylethanolamine and N-tetradecanoyl phosphatidylethanolamine.

66. The method of claim 65, wherein the at least one fusogenic lipid is selected from the group consisting of N-dodecanoyl phosphatidylethanolamine.

67. The method of claim 32, wherein the liposomes of step (A) comprise at least one fusogenic lipid.

68. The method of claim 67, wherein the at least one fusogenic lipid is selected from the group consisting of N-acyl phosphatidylethanolamine.

69. The method of claim 68, wherein the at least one fusogenic lipid is selected from the group consisting of N-decanoyl phosphatidylethanolamine, N-dodecanoyl phosphatidylethanolamine and N-tetradecanoyl phosphatidylethanolamine.

70. The method of claim 69, wherein the at least one fusogenic lipid is selected from the group consisting of N-dodecanoyl phosphatidylethanolamine.

71. The method of claim 53, wherein the liposomes of step (A) comprise at least one fusogenic lipid.

72. The method of claim 71, wherein the at least one fusogenic lipid is selected from the group consisting of N-acyl phosphatidylethanolamine.

73. The method of claim 72, wherein the at least one fusogenic lipid is selected from the group consisting of N-decanoyl phosphatidylethanolamine, N-dodecanoyl phosphatidylethanolamine and N-tetradecanoyl phosphatidylethanolamine.

74. The method of claim 73, wherein the at least one fusogenic lipid is selected from the group consisting of N-dodecanoyl phosphatidylethanolamine.

75. The method of claim 2, wherein aqueous medium V is mixed in increments with the gel or the liquid containing gel particles in step (C), wherein the increments are up to about 100% of the weight of the gel or the liquid containing gel particles before the gel or the liquid is mixed with any aqueous medium V.

76. The method of claim 75, wherein the increments are up to about 80% of the weight of the gel or the liquid containing gel particles before the gel or the liquid is mixed with any aqueous medium V.

77. The method of claim 76, wherein the increments are up to about 60% of the weight of the gel or the liquid containing gel particles before the gel or the liquid is mixed with any aqueous medium V.

78. The method of claim 77, wherein the increments are up to about 40% of the weight of the gel or the liquid containing gel particles before the gel or the liquid is mixed with any aqueous medium V.

79. The method of claim 78, wherein the increments are up to about 20% of the weight of the gel or the liquid containing gel particles before the gel or the liquid is mixed with any aqueous medium V.

80. The method of claim 79, wherein the increments are up to about 10% of the weight of the gel or the liquid containing gel particles before the gel or the liquid is mixed with any aqueous medium V.

81. The method of claim 80, wherein the increments are up to about 5% of the weight of the gel or the liquid containing gel particles before the gel or the liquid is mixed with any aqueous medium V.

82. The method of claim 81, wherein the increments are up to about 1% of the weight of the gel or the liquid containing gel particles before the gel or the liquid is mixed with any aqueous medium V.

83. The method of claim 82, wherein the increments are up to about 0.5% of the weight of the gel or the liquid containing gel particles before the gel or the liquid is mixed with any aqueous medium V.

84. The method of claim 83, wherein the increments are up to about 0.1% of the weight of the gel or the liquid containing gel particles before the gel or the liquid is mixed with any aqueous medium V.

85. The method of claim 80, wherein the increments are from about 0.001% to about 10% of the weight of the gel or the liquid containing gel particles before the gel or the liquid is mixed with any aqueous medium V.

86. The method of claim 85, wherein the increments are from about 0.001% to about 5% of the weight of the gel or the liquid containing gel particles before the gel or the liquid is mixed with any aqueous medium V.

87. The method of claim 86, wherein the increments are from about 0.001% to about 1% of the weight of the gel or the liquid containing gel particles before the gel or the liquid is mixed with any aqueous medium V.

88. The method of claim 32, wherein aqueous medium V is mixed in increments with the gel or the liquid containing gel particles in step (C), wherein the increments are up to about 100% of the weight of the gel or the liquid containing gel particles before the gel or the liquid is mixed with any aqueous medium V.

89. The method of claim 88, wherein the increments are up to about 80% of the weight of the gel or the liquid containing gel particles before the gel or the liquid is mixed with any aqueous medium V.

90. The method of claim 89, wherein the increments are up to about 60% of the weight of the gel or the liquid containing gel particles before the gel or the liquid is mixed with any aqueous medium V.

91. The method of claim 90, wherein the increments are up to about 40% of the weight of the gel or the liquid containing gel particles before the gel or the liquid is mixed with any aqueous medium V.

92. The method of claim 91, wherein the increments are up to about 20% of the weight of the gel or the liquid containing gel particles before the gel or the liquid is mixed with any aqueous medium V.

93. The method of claim 92, wherein the increments are up to about 10% of the weight of the gel or the liquid containing gel particles before the gel or the liquid is mixed with any aqueous medium V.

94. The method of claim 93, wherein the increments are up to about 5% of the weight of the gel or the liquid containing gel particles before the gel or the liquid is mixed with any aqueous medium V.

95. The method of claim 94, wherein the increments are up to about 1% of the weight of the gel or the liquid containing gel particles before the gel or the liquid is mixed with any aqueous medium V.

96. The method of claim 95, wherein the increments are up to about 0.5% of the weight of the gel or the liquid containing gel particles before the gel or the liquid is mixed with any aqueous medium V.

97. The method of claim 96, wherein the increments are up to about 0.1% of the weight of the gel or the liquid containing gel particles before the gel or the liquid is mixed with any aqueous medium V.

98. The method of claim 93, wherein the increments are from about 0.001% to about 10% of the weight of the gel or the liquid containing gel particles before the gel or the liquid is mixed with any aqueous medium V.

99. The method of claim 98, wherein the increments are from about 0.001% to about 5% of the weight of the gel or the liquid containing gel particles before the gel or the liquid is mixed with any aqueous medium V.

100. The method of claim 99, wherein the increments are from about 0.001% to about 1% of the weight of the gel or the liquid containing gel particles before the gel or the liquid is mixed with any aqueous medium V.

101. The method of claim 53, wherein aqueous medium V is mixed in increments with the gel or the liquid containing gel particles in step (C), wherein the increments are up to about 100% of the weight of the gel or the liquid containing gel particles before the gel or the liquid is mixed with any aqueous medium V.

102. The method of claim 101, wherein the increments are up to about 80% of the weight of the gel or the liquid containing gel particles before the gel or the liquid is mixed with any aqueous medium V.

103. The method of claim 102, wherein the increments are up to about 60% of the weight of the gel or the liquid containing gel particles before the gel or the liquid is mixed with any aqueous medium V.

104. The method of claim 103, wherein the increments are up to about 40% of the weight of the gel or the liquid containing gel particles before the gel or the liquid is mixed with any aqueous medium V.

105. The method of claim 104, wherein the increments are up to about 20% of the weight of the gel or the liquid containing gel particles before the gel or the liquid is mixed with any aqueous medium V.

106. The method of claim 105, wherein the increments are up to about 10% of the weight of the gel or the liquid containing gel particles before the gel or the liquid is mixed with any aqueous medium V.

107. The method of claim 106, wherein the increments are up to about 5% of the weight of the gel or the liquid containing gel particles before the gel or the liquid is mixed with any aqueous medium V.

108. The method of claim 107, wherein the increments are up to about 1% of the weight of the gel or the liquid containing gel particles before the gel or the liquid is mixed with any aqueous medium V.

109. The method of claim 108, wherein the increments are up to about 0.5% of the weight of the gel or the liquid containing gel particles before the gel or the liquid is mixed with any aqueous medium V.

110. The method of claim 109, wherein the increments are up to about 0.1% of the weight of the gel or the liquid containing gel particles before the gel or the liquid is mixed with any aqueous medium V.

111. The method of claim 106, wherein the increments are from about 0.001% to about 10% of the weight of the gel or the liquid contning gel particles before the gel or the liquid is mixed with any aqueous medium V.

112. The method of claim 111, wherein the increments are from about 0.001% to about 5% of the weight of the gel or the liquid containing gel particles before the gel or the liquid is mixed with any aqueous medium V.

113. The method of claim 112, wherein the increments are from about 0.001% to about 1% of the weight of the gel or the liquid containing gel particles before the gel or the liquid is mixed with any aqueous medium V.

114. The method of claim 53, wherein the at least one biologically active substance is selected from the group consisting of proteins and antigens structurally sensitive to dehydration.

115. The method of claim 114, wherein the at least one biologically active substance is a tetanus toxoid.

116. The method of claim 2, wherein the liposomes provided in step (A)(a), (A)(D), (A)(c), (A)(d) or (A)(f) comprise at least one charged lipid, the liposomes in step (A)(e) are formed in the presence of at least one charged lipid and the at least one biologically active substance, or at least one charged lipid is added in step (B), wherein the at least one charged lipid is a lipid chaving a net negative or positive charge.

117. The method of claim 116, wherein the at least one charged lipid is selected from the group consisting of N-acyl phosphatidylethanolamine, phosphatidylserine, phosphatidylinositol, phosphatidylglycerol, diphosphatidylglycerol and phosphatidic acid.

118. The method of claim 116, wherein the at least one charged lipid is liposome forming.

119. The method of claim 32, wherein the liposomes provided in step (A)(a), (A)(b), (A)(c), (A)(d) or (A)(f) comprise at least one charged lipid, the liposomes in step (A)(e) are formed in the presence of at least one charged lipid and the at least one biologically active substance, or at least one charged lipid is added in step (B), wherein the at least one charged lipid is a lipid chaving a net negative or positive charge.

120. The method of claim 119, wherein the at least one charged lipid is selected from the group consisting of N-acyl phosphatidylethanolamine, phosphatidylserine, phosphatidylinositol, phosphatidylglycerol, diphosphatidylglycerol and phosphatidic acid.

121. The method of claim 119, wherein the at least one charged lipid is liposome forming.

122. The method of claim 53, wherein the liposomes provided in step (A)(a), (A)(b), (A)(c), (A)(d) or (A)(f) comprise at least one charged lipid, the liposomes in step (A)(e) are formed in the presence of at least one charged lipid and the at least one biologically active substance, or at least one charged lipid is added in step (B), wherein the at least one charged lipid is a lipid chaving a net negative or positive charge.

123. The method of claim 122, wherein the at least one charged lipid is selected from the group consisting of N-acyl phosphatidylethanolamine, phosphatidylserine, phosphatidylinositol, phosphatidylglycerol, diphosphatidylglycerol and phosphatidic acid.

124. The method of claim 123, wherein the at least one charged lipid is liposome forming.

125. The method of claim 2, wherein the amount of lipid in the gel or the liquid containing gel particles of step (B) ranges from 1% by weight of the gel or the liquid containing gel particles to the hydration limit of the lipid in water, wherein the “hydration limit” is the maximum amount of lipid in a given amount of water that would keep the lipid in a liposomal state.

126. The method of claim 2, wherein the amount of lipid in the gel or the liquid containing gel particles of step (B) ranges from about 5% to about 95% by weight of the gel or the liquid containing gel particles.

127. The method of claim 126, wherein said amount of lipid ranges from about 10% to about 95% by weight of the gel or the liquid containing gel particles.

128. The method of claim 127, wherein said amount of lipid ranges from about 15% to about 95% by weight of the gel or the liquid containing gel particles.

129. The method of claim 128, wherein said amount of lipid ranges from about 20% to about 95% by weight of the gel or the liquid containing gel particles.

130. The method of claim 129, wherein said amount of lipid ranges from about 30% to about 95% by weight of the gel or the liquid containing gel particles.

131. The method of claim 130, wherein said amount of lipid ranges from about 40% to about 95% by weight of the gel or the liquid containing gel particles.

132. The method of claim 131, wherein said amount of lipid ranges from about 50% to about 95% by weight of the gel or the liquid containing gel particles.

133. The method of claim 132, wherein said amount of lipid ranges from about 60% to about 95% by weight of the gel or the liquid containing gel particles.

134. The method of claim 133, wherein said amount of lipid ranges from about 70% to about 95% by weight of the gel or the liquid containing gel particles.

135. The method of claim 32, wherein the amount of lipid in the gel or the liquid containing gel particles of step (B) ranges from 1% by weight of the gel or the liquid containing gel particles to the hydration limit of the lipid in water, wherein the “hydration limit” is the maximum amount of lipid in a given amount of water that would keep the lipid in a liposomal state.

136. The method of claim 32, wherein the amount of lipid in the gel or the liquid containing gel particles of step (B) ranges from about 5% to about 95% by weight of the gel or the liquid containing gel particles.

137. The method of claim 136, wherein said amount of lipid ranges from about 10% to about 95% by weight of the gel or the liquid containing gel partcles.

138. The method of claim 137, wherein said amount of lipid ranges from about 15% to about 95% by weight of the gel or the liquid containing gel particles.

139. The method of claim 138, wherein said amount of lipid ranges from about 20% to about 95% by weight of the gel or the liquid containing gel particles.

140. The method of claim 139, wherein said amount of lipid ranges from about 30% to about 95% by weight of the gel or the liquid containing gel particles.

141. The method of claim 140, wherein said amount of lipid ranges from about 40% to about 95% by weight of the gel or the liquid containing gel particles.

142. The method of claim 141, wherein said amount of lipid ranges from about 50% to about 95% by weight of the gel or the liquid containing gel particles.

143. The method of claim 142, wherein said amount of lipid ranges from about 60% to about 95% by weight of the gel or the liquid containing gel particles.

144. The method of claim 143, wherein said amount of lipid ranges from about 70% to about 95% by weight of the gel or the liquid containing gel particles.

145. The method of claim 53, wherein the amount of lipid in the gel or the liquid containing gel particles of step (B) ranges from 1% by weight of the gel or the liquid containing gel particles to the hydration limit of the lipid in water, wherein the “hydration limit” is the maximum amount of lipid in a given amount of water that would keep the lipid in a liposomal state.

146. The method of claim 53, wherein the amount of lipid in the gel or the liquid containing gel particles of step (B) ranges from about 5% to about 95% by weight of the gel or the liquid containing gel particles.

147. The method of claim 146, wherein said amount of lipid ranges from about 10% to about 95% by weight of the gel or the liquid containing gel particles.

148. The method of claim 147, wherein said amount of lipid ranges from about 15% to about 95% by weight of the gel or the liquid containing gel particles.

149. The method of claim 148, wherein said amount of lipid ranges from about 20% to about 95% by weight of the gel or the liquid containing gel particles.

150. The method of claim 149, wherein said amount of lipid ranges from about 30% to about 95% by weight of the gel or the liquid containing gel particles.

151. The method of claim 150, wherein said amount of lipid ranges from about 40% to about 95% by weight of the gel or the liquid containing gel particles.

152. The method of claim 151, wherein said amount of lipid ranges from about 50% to about 95% by weight of the gel or the liquid containing gel particles.

153. The method of claim 152, wherein said amount of lipid ranges from about 60% to about 95% by weight of the gel or the liquid containing gel particles.

154. The method of claim 153, wherein said amount of lipid ranges from about 70% to about 95% by weight of the gel or the liquid containing gel particles.
